(19) United States
(12) Patent Application Publication
(10) Pub. No.: US 2007/0261471 A1
(43) Pub. Date: Nov. 15, 2007

(54) Soot sensor

(76) Inventors: Tomonori Kondo, Komaki-shi (JP); Norihiko Nadanami, Inuyama-shi (JP); Daisuke Komatsu, Nisshin-shi (JP); Hitoshi Yokoi, Ama-gun (JP); Masato Katsuta, Komaki-shi (JP)

Correspondence Address: STITES & HARBISON PLLC, 1199 NORTH FAIRFAX STREET, SUITE 900, ALEXANDRIA, VA, 22314, US

(21) Appl. No.: 11/797,802

(22) Filed: May 8, 2007

(30) Foreign Application Priority Data
May 9, 2006 (JP) P.2006-129771

(51) Int. Cl. G01N 33/497; G01N 7/00; G01N 29/02

(52) U.S. Cl. 73/23.2; 73/19.01

(57) ABSTRACT

A soot sensor includes an insulator having a through-hole and a center electrode provided in the through-hole of the insulator so that a leading end of the center electrode protrudes from a leading end of the insulator and faces a discharge gap. A heating member is embedded in the insulator, and the distance between the heating member and the leading end of the center electrode is at least 10 mm.

### BACKGROUND OF THE INVENTION

1. Field of the Invention

The present invention relates to a soot sensor.

2. Description of the Related Art

There is disclosed in JP-UM-A-64-50355 a detecting section provided in a smoke detector which serves as a conventional soot sensor. The detecting section of the smoke detector accommodates a rod-like or stick-shaped center electrode within a housing through an insulator, exposes the leading end of the center electrode to the outside, places an outer electrode connecting to the housing around the leading end of the center electrode through a gap and exposes the center electrode and outer electrode of the detecting section into exhaust gas, wherein spark discharge occurs when high voltage is applied between the center electrode and the outer electrode. Then, based on the principle that as the amount of soot in exhaust gas increases, the degree of reduction of the voltage (which is equivalent to discharge voltage) upon occurrence of the spark discharge increases, the presence of soot and/or the amount of soot in the exhaust gas is/are detected based on the discharge voltage.

In a soot sensor having this construction, the accuracy of the soot detection may be reduced by soot adhered to the insulator. Furthermore, the spark discharge itself is not enough to remove the adhered soot, and the soot is desirably destroyed by a heater.

On the other hand, the soot adhered to the center electrode and/or outer electrode can be removed or destroyed by a heater in the detecting section as disclosed in W. D. E. Allan, R. D. Freeman, G. R. Pucher, D. Faux and M. F. Bardon, “DEVELOPMENT OF A SMOKE SENSOR FOR DIESEL ENGINES, Royal Military College of Canada, D. P. Gardiner, Nexum Research Corporation, p. 220, Powertrain & Fluid Systems Conference, Oct. 27-30, 2003.

Providing a heater in the detecting section as described above presents other problems. For example, this may reduce the discharge voltage even for an exhaust gas where there is little or no soot. Moreover, the discharge voltage is somewhat reduced even by exposing the center electrode and outer electrode to exhaust gas containing soot. This causes a problem in that it is difficult to detect the presence of soot or the amount of soot from the discharge voltage.

### SUMMARY OF THE INVENTION

According to one aspect of the background of the invention, the above-described problem has been studied in detail. According to this study, since soot is a substance made from conductive particles, which are carbon particles, the soot may be responsible for the reduction of discharge voltage.

On the other hand, since the discharge voltage is reduced even by exhaust gas containing very little soot as described above, particles responsible for the conductivity of ions, i.e., conductive particles, exerting substantially the same effect as that of soot may exist therein, in addition to soot.

As a result of various studies from this point of view, the occurrence of the problems described above can be prevented by properly defining the position at which the heater is provided in the detecting section, taking into consideration the relationship thereof with a discharge area between the center electrode and the outer electrode.

Accordingly, an important aspect of the present invention is based on this point of view, and it is an object of the invention to appropriately position a heater for burning out soot on the insulator, while preventing the negative influence by conductive particles, apart from soot.

In order to achieve the above object and other objects, a soot sensor according to a first aspect of the invention is provided including:

an insulator having a through-hole;

a center electrode provided in the through-hole of the insulator so that a leading end of the center electrode protrudes from a leading end of the insulator and faces a discharge gap; and

a heating member embedded in the insulator.

In the soot sensor of this aspect of the invention, the distance spacing between a leading (discharge gap side) end of the heating member and the leading (discharge gap side) end of the center electrode is at least 10 mm. This distance is measured along the axis of the center electrode.

With a distance between the leading end of the heating member and the leading end of the center electrode of at least 10 mm, as described above, when a high voltage is applied to the center electrode, the high voltage is also applied between the heating member of the heater and the center electrode, and a discharge occurs between the heating member and the center electrode. Although this generates particles responsible for conductivity (conductive particles) such as ions, the conductive particles do not move to the discharge gap. Therefore, when soot does not exist in the discharge gap, the discharge voltage of the discharge gap is not influenced by the conductive particles. This means that the discharge voltage of the discharge gap is only reduced by soot. As a result, with this soot sensor, soot can be detected with high precision without any influence from conductive particles.

According to a second aspect of the invention, in the soot sensor according to the first aspect of the invention, the length along the surface of the insulator from the leading end of the insulator to the leading end of the heating member on the discharge gap side is between about 3 mm to 12 mm.

By having a lower limit value of about 3 mm as the length along the surface of the insulator from the leading end of the insulator on the discharge gap side to the leading end of the heating member on the discharge gap side, the heating member is not located too close to the discharge gap. As a result, any occurrence of short circuiting (shorting) or discharging between the heating member and the center electrode can be prevented in advance. In particular, the lower limit value of the length takes into consideration the fact that shorting or discharging may readily occur through the surface of an insulator. By specifying an upper limit value of 12 mm with respect to the length along the surface of the insulator from the end of the insulator on the discharge gap side to the leading end of the heating member on the discharge gap side, the undesired deposition of soot on the discharge gap side of the insulator can be prevented in advance.

As a result, the occurrence of shorting or discharge between the heating member and the center electrode and the improper deposition of soot on the discharge gap side of the insulator as described above can be prevented in advance, and the operational effects of the invention according to the first aspect of the invention can be additionally obtained.

In accordance with a third aspect of the invention, in a soot sensor according to any one of the first and/or second aspects of the invention, the insulator may have a thickness of from 0.7 mm to 3 mm at a part disposed between the heating member and the center electrode.

A thickness of larger than about 0.7 mm at the part of the insulator between the heating member and the center electrode can prevent discharging in the thickness direction since otherwise the part of the insulator between the heating member and the center electrode will be excessively thin. On the other hand, a thickness of smaller than about 3 mm at the part of the insulator between the heating member and the center electrode will not increase the heat capacity which would otherwise occur if the part of the insulator between the heating member and the center electrode is excessively thick.

As a result, discharge in the direction of the thickness and an improper increase in the heat capacity of the part of the insulator between the heating member and the center electrode can be prevented, and the operational effects of the invention according to the first aspect can be also obtained.

According to a fourth aspect of the invention, the soot sensor according to any one of the first to third aspects of the invention may further include a metal housing surrounding the periphery of the insulator. In this aspect of the invention, the leading end of the insulator is positioned within the metal housing.

Positioning the leading end of the insulator within the metal housing prevents the leading end of the insulator from being readily exposed to soot from the outside of the metal housing. As a result, the operational effects of the invention according to the first aspect can be improved.

In accordance with a fifth aspect of the invention, the soot sensor according to any one of the first to fourth aspects of the invention includes an outer electrode connected to the metal housing and having a tip section disposed opposite the leading end of the center electrode across a discharge gap.

According to a sixth aspect of the invention, in the soot sensor according to any one of the first to fifth aspects, the center electrode and the heater use the metal housing as a common ground.

Thus, with this common arrangement, the center electrode and the heater do not require respective grounds but can share a single, common ground. As a result, the ground structure of the soot sensor can be simplified when the operational effect of the invention according to any one of the previously described aspects is obtained.

Further features and advantages of the present invention will be set forth in, or apparent from, the detailed description of preferred embodiments thereof which follows.

### BRIEF DESCRIPTION OF THE DRAWINGS

FIG. 1 is a partially cut away side elevation view illustrating a first embodiment of a spark plug type soot sensor according to the invention.

FIG. 2 is a partially cut away elevation view similar to that of FIG. 1 showing a first embodiment of the invention.

FIG. 3 is an enlarged partially cut away plan view showing the heater in FIG. 1.

FIG. 4 is a graph showing a relationship between (i) soot sensitivities of a soot sensor according to the first embodiment and (ii) predetermined distances each corresponding to distance between the leading end of an outer heat element section of the heater and the leading end portion of a tip section of the center electrode.

FIG. 5 is a side elevation view, partially in cross section, illustrating a second embodiment of a spark plug type soot sensor according to the invention.

FIG. 6 is a partially cut away plan view illustrating a principal or main part of a third embodiment of the invention.

FIG. 7 is a side elevation view, partially in cross section, showing a fourth embodiment of the invention.

### DETAILED DESCRIPTION OF PREFERRED EMBODIMENTS

With reference to drawings, embodiments of the invention will be described below.

### First Embodiment

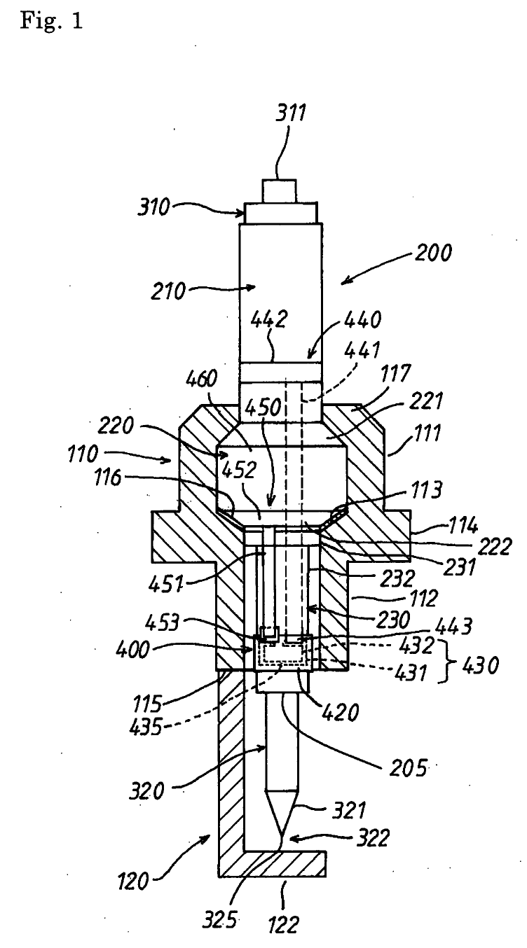

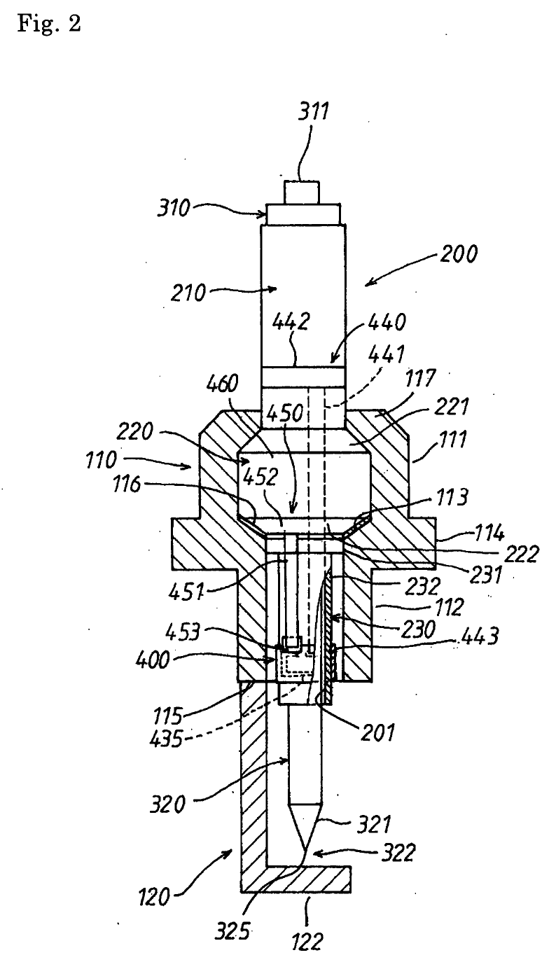

FIG. 1 shows a first embodiment of a spark plug type soot sensor according to the invention. This soot sensor includes an insulator 200 having a through-hole 201 (cf. FIG. 2); a center electrode 320 provided in the through-hole 201 of the insulator 200 so that a leading end 325 of the center electrode 320 protrudes from a leading end 205 of the insulator 200 and faces a discharge gap 322; a heating member 430 embedded in the insulator 200; a metal housing 110 surrounding the periphery of the insulator 200; and an outer electrode 120 connected to the metal housing 110.

The metal housing 110 has a base end side section 111, a leading end side section 112, and a flange section 114 located between the base end side section 111 and the leading end side section 112. The base end side section 111, leading end side section 112 and flange section 114 are preferably formed by a metal steel material and are integral, and arranged coaxially and cylindrically, as shown in FIG. 1.

The leading end side section 112 has a smaller inside diameter than that of the base end side section 111. The inner circumferential surface of the flange section 114 is widened toward the inner circumferential surface of the base end side section 111 to have an annular stepped portion 113.

A base end of the outer electrode 120 is connected to a part of a leading end 115 of the section 110. The tip section 122 faces toward a tip section of the center electrode 321. According to the first embodiment, the outer electrode 120 is used as a negative pole. The outer electrodes 120 preferably contains a material such as is used for a spark plug such as a nickel alloy, iridium, platinum, tungsten and SUS steel.

The insulator 200 has a base end side part 210, a flange part 220 and a leading end side part 230. The base end side part 210, flange part 220 and leading end side part 230 are formed integrally, and arranged coaxially and cylindrically, as shown in FIG. 1 and made from an electric insulating material such as ceramics.

The flange part 220 is formed to have a larger outside diameter than those of the base end side part 210 and leading end side part 230 between the base end side part 210 and the leading end side part 230. The middle part includes shoulder portions 221 and 222.

As shown in FIG. 1, the leading end side part 230 includes the large diameter part 231, a small diameter part 232, and a heater section 400 formed around the small diameter part. The small diameter part 232 is slightly narrowed from the end connected to the large diameter part 231 to the leading end 205 of the insulator 200.

The leading end side part 230 is partially arranged in the leading end side section 112 of the metal housing 110. The heater section 400 is also arranged within the metal housing 110. The flange part 220 is fitted inside of the base end side section 111 of the metal housing 110, and the shoulder portion 222 is seated on the stepped portion 113 of the leading end side section 112 through means of a metallic ring-shaped collar 116. Thus, the insulator 200 is supported coaxially within the metal housing 110. The large diameter part 231 of the insulator 200 is fitted in the leading end side section 112 of the metal housing 110. The metal housing 110 is crimped to the shoulder portion 221 of the flange part 220 of the insulator 200 at the crimp portion 117 of the metal housing 110.

The center electrode 320 is arranged in the through-hole of the insulator 200. The base section of the center electrode is located in the leading end side part 230 of the insulator 200. The center electrode 320 has a tip section 321, which has a conical shape as shown in FIG. 1. The vertex angle of the tip section 321 is about 60 degrees, for example. The outside diameter of the part of the center electrode 320, excluding the tip section 321, is 2 mm, in one illustrative example. The center electrode 320 is used as a positive electrode.

The tip section 321 of the center electrode 321 faces toward the tip section 122 of the outer electrode 120 through the discharge gap 322 (having a width of, e.g., 0.5 mm) in the vertical direction. According to this embodiment, the tip sections 122 and 321 face toward each other through the discharge gap 322 at which a discharge is generated between them.

The gap between the part of the center electrode 320 excluding the tip section 321 and the outer electrode 120 in horizontal direction is larger than the discharge gap 322 in axial direction in order to allow the discharge between the tip sections 122 and 321.

The center electrode 320 is coaxially connected to a rod-shaped conductive member 310 which is located in the through hole of the base end side part 210 of the insulator. The conductive member 310 includes the base end portion 311 for connecting the center electrode 320 to a high voltage circuit (not shown).

When a predetermined high voltage is applied between the outer electrode 120 and the center electrode 320 from the high voltage circuit in the soot sensor, the outer electrode 120 and the center electrode 320 discharge between the tip sections 122 and 321 facing toward each other (that is, in the discharge gap 322). In this case, the voltage to be applied between the tip sections 122 and 321 is detected as the voltage occurring upon discharge (which hereinafter is referred to as the discharge voltage). The discharge voltage is reduced when soot exists between the tip sections 122 and 321.

According to the first embodiment, the predetermined high voltage is dielectrically defined as a voltage of 10 kV, for example, that breaks down the air between the tip sections 122 and 321 forming the spark gap, and causes a discharge between the tip sections 122 and 321.

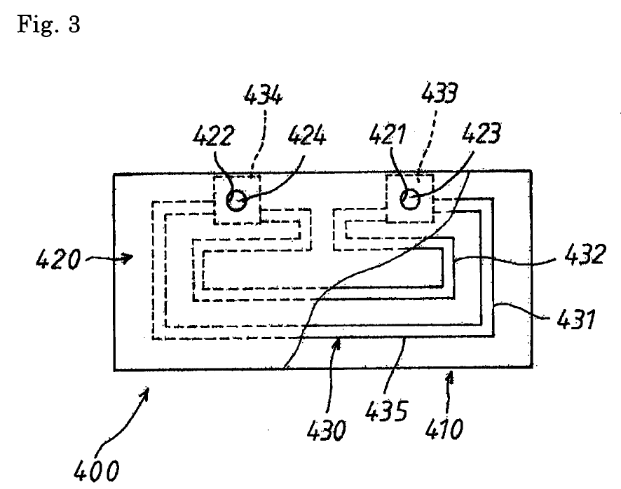

The heater section 400 prevents shorting or discharge due to soot in the tip sections 122 and 321 by heating the lead end side part 230 of the insulator so as to burn off soot deposited on the outer surface of the lead end side part 230. The heater section 400 includes two alumina sheets 410 and 420 and a heating member 430 sandwiched between the alumina sheets 410 and 420, as shown in FIG. 3.

The heating member 430 has an outer heat element section 431 of a band shape, an inner heat element section 432 in a band shape, and positive and negative electrode pads 433 and 434. The heat element sections 431 and 432 and electrode pads 433 and 434 are formed by printing and firing a platinum paste on the inner surface of the alumina sheet 410 in predetermined patterns as shown in FIG. 3.

The alumina sheet 420 has through-holes 421 and 422. The through hole 421 is positioned at the center part of the positive side electrode pad 433 while the through-hole 422 is positioned at the center part of the negative side electrode pad 434.

In the heater section 400 having this construction, when soot is deposited in the insulator 200 to a degree that prevents proper discharge between the tip sections 122 and 321, the heating member 430 is heated by the application of a heater voltage (of 15 V, for example) from a heater driving circuit (not shown) and thus performs heat-cleaning of the heating member 430. It is noted that the heat-cleaning is performed under conditions wherein the application of a high voltage to the tip sections 122 and 321 from the high voltage circuit is terminated.

According to this embodiment, the surface length, which can be defined along the surface of the insulator 200, between the leading end 435 of the heating member 430 and the leading end 205 of the insulator 200 is set to be between about 3 mm and 12 mm (and 4 mm in the first embodiment). Since the surface length is 3 mm or more, a short circuit or discharge between the heating member 430 and the center electrode 320 can be prevented. Furthermore, since the surface length is 12 mm or less, soot deposited on the surface of the insulator 200 can be effectively burned off by the heater section 400.

The soot sensor, as shown in FIG. 1, includes positive and negative side leads 440 and 450 for the heater section 400 and a glass layer 460 for the positive and negative leads 440 and 450.

The positive side lead 440 has an axial lead section 441 and a circumferential lead section 442. The axial lead section 441 extends from the leading end portion 443 of the axial lead section 441, which is provided on the electrode pad 433 of the heater section 400, to the circumferential lead section 442. The circumferential lead section 442 is provided circumferentially on the outer circumferential surface of the base end side part 210 of the insulator 200.

The negative side lead 450 is provided on the glass layer 460, which covers the outer circumferential surface of the insulator 200. The negative side lead 450 has an axial lead section 451 and a circumferential lead section 452.

The axial lead section 451 extends from the leading end portion 453, which is provided on the electrode pad 434 of the heater section 400, to the circumferential lead section 452. The circumferential lead section 452 extends circumferentially along the shoulder portion 222 of the flange part 220 of the insulator 200.

The glass layer 460 extends in a range or area extending from the base end the heater section 400 to the circumferential lead section 442 so that the circumferential lead section 442 is exposed, i.e., extends outwards from the glass layer.

Since the negative side lead 450 is formed on the glass layer 460, the negative side lead 450 is electrically insulated from the positive side lead 440 by the glass layer 460.

Next, there will be described a construction for enabling discharge by the soot sensor without the influence of particles (excluding soot) responsible for the conductivity described above.

The axial distance between the leading end 435 of the heat element 431 and the leading end 325 of the tip section 321 of the center electrode 320 is set to be 10 mm or more, i.e. 25 mm in the first embodiment.

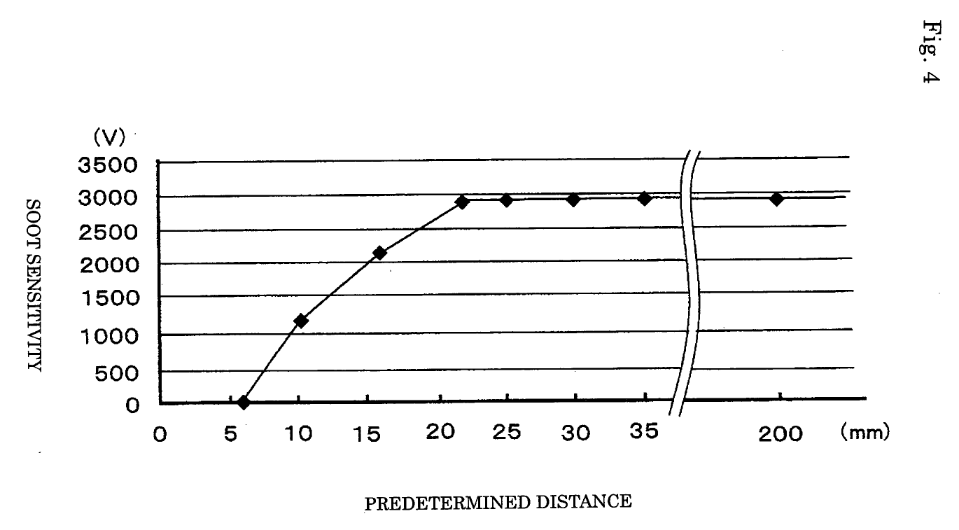

It is noted that measuring the relationship between the soot sensitivity of the soot sensor and the axial distance between the leading end 435 of the heating member 431 and the leading end 325 of the center electrode 320 results in the graph as shown in FIG. 4. The axial distance of the samples shown in FIG. 4 are 6 mm (comparative sample), 10 mm, 16 mm, 22 mm, 30 mm, 35 mm and 200 mm.

The measurement used a GFG-1000 model soot generator (PALAS, German) which generates soot at 3 mg/m³. The measuring circuit is configured to apply a high voltage between the center electrode and the outer electrode from the high voltage circuit, and to measure the discharge voltage occurring between the center electrode and outer electrode by using an oscilloscope. A measurement of soot sensitivity is obtained for each of the soot sensors by performing the measurement 100 times and calculating the average value of the measurement results.

The soot sensitivity is defined by the difference between the discharge voltage occurring between the tip sections 122 and 321 when soot is not present between the tip sections 122 and 321 and the discharge voltage occurring between the tip sections 122 and 321 when soot is present between the tip sections 122 and 321.

The graph of FIG. 4 shows that the soot sensitivity of the soot sensor in the comparison example (wherein the axial distance equal to 6 mm) is zero V. This may be due to a large influence of ions as in the following.

Here, the heater section 400 is connected to the metal housing 110 like the outer electrode 120. The resistance value of the heating member 430 of the heater section 400 is about several Ω. For this reason, the heater section 400 may have a substantially equal potential (ground potential) to that of the metal housing 110.

Therefore, when the predetermined high voltage is applied between the outer electrode 120 (metal housing 110) and the center electrode 320, the predetermined high voltage is also applied between the heater section 400 (metal housing 110) and the center electrode 320 through the insulator 200 and the space between the insulator 200 and the center electrode 320. As a result, a discharge occurs between the leading end side part 230 of the insulator 200 and the center electrode 320.

When the discharge changes to Corona discharge, for example, the Corona discharge acts between the heating member 430 of the heater section 400 and the center electrode 320 through the circumference of the leading end side part 230 of the insulator 200. Thus, any gas present between the leading end side part 230 of the insulator 200 and the center electrode 320 is ionized.

The generated ions are assumed to move from the inside of the leading end side part 230 of the insulator 200 toward the electrode 321 side of the center electrode 320 and electrically act as particles responsible for the conductivity between the tip sections 321 and 122, similarly to soot.

This means that the atmosphere between the tip sections 321 and 122 may induce a similar discharge phenomenon to that in the atmosphere containing soot even when the atmosphere between the tip sections 321 and 122 does not contain soot but rather only the ions described above. In other words, the presence of soot is improperly detected from the presence of the ions, even when soot is not present. As a result, the discharge voltage is not differentiated based on the presence of soot, which obviously reduces the accuracy of the detection of soot.

The graph of FIG. 4 also shows that the soot sensitivity sequentially increases to 1200 V, 2200 V, about 2900 V, respectively and in the soot sensors with the predetermined distances equal to 10 mm, 16 mm and 22 mm, respectively. This may be due to the influence of ions based on the estimation, but the degree of the influence by the ions may decrease as the predetermined distance increases.

In the soot sensors with the predetermined distances equal to 30 mm, 35 mm and 200 mm, the graph of FIG. 4 shows that the soot sensitivities are constant at about 2900 V similarly to the soot sensor with the predetermined distance equal to 22 mm. This may be because it is not influenced by the ions based on the estimation above.

According to the results of this study, if the predetermined distance falls in the range from at least close to 10 mm to 200 mm, the soot sensor should have the appropriate soot sensitivity since the ions above cannot move to the discharge gap 332.

A limit on the predetermined distance to equal to or smaller than 200 mm is appropriate since it is difficult to install the soot sensor in an exhaust pipe of the engine of a vehicle if the predetermined distance is larger than 200 mm.

According to the first embodiment having this construction, the soot sensor is mounted on an exhaust pipe of a vehicle diesel engine such that the discharge gap 322 of the soot sensor can be exposed within the exhaust pipe.

However, in the soot sensor, the base end portion 311 of the conductive member 310 and the metal housing 110 (ground) are connected to the positive and negative side output terminals of the high voltage circuit. Also in the soot sensor, the circumferential lead section 442 of the positive side lead 440 and the metal housing 110 (ground) are connected to the positive and negative side output terminals of a heater driving section (not shown).

In this state, when the diesel engine starts, the discharge gap 322 of the soot sensor is exposed to exhaust gas (corresponding to the atmosphere described above) emitted into the inside of the exhaust pipe of the diesel engine.

When the high voltage circuit generates the high voltage after a certain period of time, the high voltage is applied between the base end portion 311 of the conductive member 310 and the metal housing 110. This means that the high voltage is applied to the center electrode 320 by employing the outer electrode 120 as a negative pole.

If the exhaust gas flowing within the exhaust pipe does not contain soot, the air between the tip section 122 of the outer electrode 120 and the tip section 321 of the center electrode 320 (that is, the air in the discharge gap 322) is broken down by the high voltage. As a result, a discharge occurs between the tip sections 122 and 321.

On the other hand, if the exhaust gas flowing within the exhaust pipe contains soot, the air between the tip section 122 of the outer electrode 120 and the tip section 321 of the center electrode 320 contains soot and this air is then broken down. As a result, a discharge occurs between the tip sections 122 and 321. However, the discharge voltage in this case is lower, by the amount equal to the density of soot, than the discharge voltage due to the breakdown by the exhaust gas not containing soot.

Therefore, if a reduced discharge voltage is detected in this way, detection of soot or the detection of the density of the soot can be achieved.

Here, as described above, a predetermined distance equal to 25 mm is defined between the leading end 435 of the heat element section 431 of the heater section 400 and the leading end 325 of the tip section 321 of the center electrode 320.

For this reason, even when a discharge occurs between the leading end side part 230 of the insulator 200 and the center electrode 320 due to the application of a high voltage (which has been applied between the outer electrode 120 and the center electrode 320), between the heater section 400 and the center electrode 320, which both are at a substantially equal potential (e.g., both are at ground potential) to that of the metal housing 110, and when ions are thus generated within the leading end side part 230 of the cylindrical 200, the ions cannot move to the discharge gap 322 and are not mixed into the exhaust gas in the discharge gap 322.

Therefore, even when the exhaust gas of the discharge gap 322 does not contain soot, the discharge voltage between the tip sections 122 and 321 is not influenced by the ions. This means that only the discharge voltage between the tip sections 122 and 321 is reduced by soot. As a result, the soot sensor can detect soot with high precision without influences of the ions.

The high precision detection provided allows control of fuel injection by a diesel engine with high precision and further allows proper detection of deterioration of a diesel particulate filter (a so-called DPF) by using the output of the detection of the soot sensor. Further, using the result of the summation of the densities of soot, which are the detection outputs of the soot sensor, allows the estimation of the proper recycling time for a DPF having collected particulate matter exhausted from a diesel engine.

Since the outer electrode 120 and the heater section 400 share the metal housing 110 as a common ground as described above, only one ground is required for the outer electrode 120 and heater section 400 in the soot sensor, instead of respective grounds for the two. Therefore, the ground structure of the soot sensor is simplified.

Based on the construction described above, a spark plug form is adopted as the form of the soot sensor. Therefore, good electric insulation of the part excluding the discharge gap 322 of the soot sensor and good wear-resistance of the center electrode 320 can be obtained.

In the detection process carried out by the soot sensor, the insulator 200 tends to improperly collect soot after a lapse of a predetermined period. For this reason, after a lapse of this predetermined period, a heater voltage is applied between the metal housing 110 and the circumferential lead section 442 of the positive side lead 440 by the heater driving circuit.

Thus, the heater voltage is applied between the terminals of the heat element sections 431 and 432 of the heater section 400. As a result, the heater section 400 generates heat in the heat element sections 431 and 432 and heats the insulator 200.

Accordingly, the soot deposited on the insulator 200 is burned off by the heater section 400. As a result, the soot sensor can properly prevent wear on and shortening of the tip sections 122 and 321 due to the deposition of soot and can accurately detect soot in a stable manner.

According to the first embodiment, the predetermined surface length above is defined as being between 3 mm and 12 mm as described above. Thus, the occurrence of shortening of or discharge between the heating member 430 of the heater section 400 and the center electrode 320, and the improper deposition of soot on the leading end side of the insulator 200 can be prevented in advance, and the various improved operational effects described above can be achieved.

FIG. 2 shows a partial cross sectional view of the leading end side part 230 of the insulator 200 according to the first embodiment of the present invention.

The thickness of a part of the insulator 200, which is sandwiched between the center electrode 320 and heating member 430, is within the range of from about 0.7 mm to 3 mm, and is, e.g., 1 mm in this embodiment.

According to the first embodiment, since the thickness of the insulator 200 is within the range of from about 0.7 mm to 3 mm, the insulator 200 can prevent a discharge through the insulator 200 in the thickness direction due to the fact that the insulator 200 is of sufficient thickness, without improperly increasing the heat capacity of the insulator.

### Second Embodiment

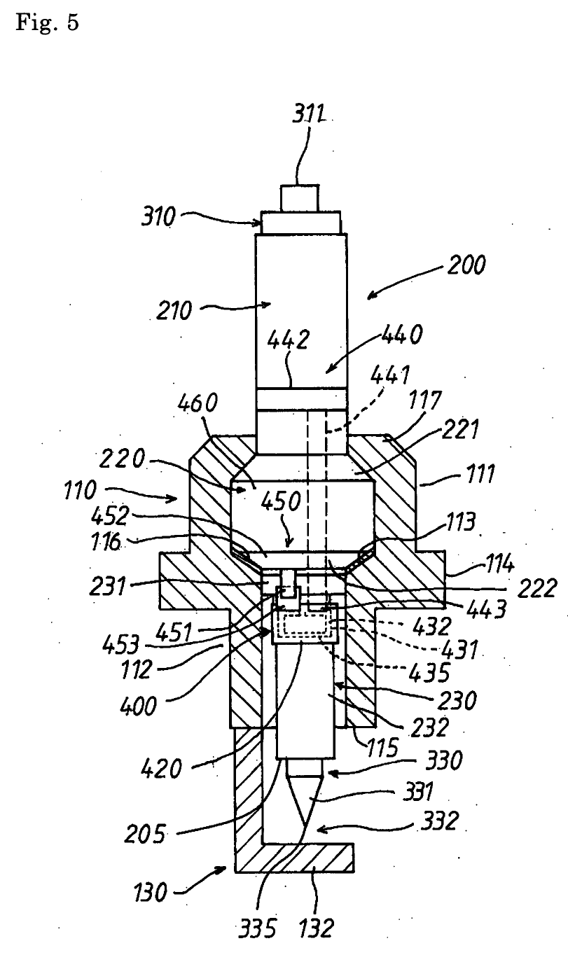

FIG. 5 shows a second embodiment of a spark plug type soot sensor according to the invention. The soot sensor of the second embodiment includes, in comparison with the first embodiment, a planar outer electrode 130 instead of the outer electrode 120, and a center electrode 330 instead of the center electrode 320.

The outer electrode 130 has a tip section 132 and a base end portion which is integrally connected to the leading end 115 of metal housing 110 as described above for the first embodiment. The tip section 132 faces toward the leading end 115 of the metal housing 110.

The center electrode 330 is coaxially connected to the conductive member 310, as described for the first embodiment, in the insulator 200. The center electrode 330 has, as shown in FIG. 5, a conical leading end portion as a tip section 331. The tip section 331 faces toward the tip section 132 of the outer electrode 130 through a discharge gap 332, similarly to the first embodiment.

However, according to the second embodiment, the extension length of the center electrode 330 from the leading end 205 of the insulator 200 is equal to about 5 mm. It is noted that the center electrode 330 is used as a positive pole similarly to the center electrode 320 according to the first embodiment. The tip sections 132 and 331 face each other through the discharge gap 332 in which a discharge takes place in the soot sensor.

According to the second embodiment, the heater section 400 is located around the small diameter part 232 of the leading end side part 230 as shown in FIG. 5. More specifically, the heater section 400 is positioned such that the axial (vertical) distance between the leading end 435 of the heat element section 431 of the heater section 400 and the leading end 335 of the center electrode 330 is about 15 mm.

According to the second embodiment, the discharge gap 332 of the soot sensor is exposed to exhaust gas emitted into the inside of the exhaust pipe of a diesel engine. Further, when a high voltage from the high voltage circuit is applied to the center electrode 330 by employing the outer electrode 130 as a negative pole, a discharge occurs between the tip sections 132 and 331.

Similarly to the first embodiment, the discharge voltage, upon discharging, is lower that that for a case without soot, by the amount of the density of the soot present between the tip sections 132 and 331. Therefore, detection of the discharge voltage which is reduced in this way allows the detection of the soot or the detection of the density of the soot.

In this embodiment, as described above, the center electrode 330 only extends from the leading end 205 of the insulator 200 by about 5 mm, but the distance between the leading end 435 of the heating member 431 of the heater section 400 and the leading end 205 of the insulator 200 is set to be between about 3 mm and 12 mm, i.e. 10 mm in the second embodiment.

For this reason, even when a discharge occurs between the leading end side part 230 of the insulator 200 and the center electrode 330 due to the application of a high voltage (which has been applied between the outer electrode 130 and the center electrode 330), between the heater section 400 and the center electrode 320, which are at a substantially equal potential (ground potential) to that of the metal housing 110, and when ions are thus generated, the ions cannot move to the discharge gap 332 and are not mixed into the exhaust gas in the discharge gap 332.

Therefore, even when the exhaust gas of the discharge gap 332 does not contain soot, the discharge voltage between the tip sections 132 and 331 is not influenced by the presence of ions. This, of course, means that the discharge voltage between the tip sections 132 and 331 is only reduced by soot. As a result, the soot sensor can detect soot with high precision without any influence from the ions.

According to this embodiment, the surface length, which can be defined along the surface of the insulator 200, between the leading end 435 of the heating member 430 and the leading end 205 of the insulator 200 is set to be between about 3 mm and 12 mm (11 mm in the first embodiment). Since the surface length is 3 mm or more, short circuiting or discharge between the heating member 430 and the center electrode 330 can be prevented. Further, since the surface length is 12 mm or less, any soot deposited on the surface of the insulator 200 can be effectively burned off by the heater section 400.

### Third Embodiment

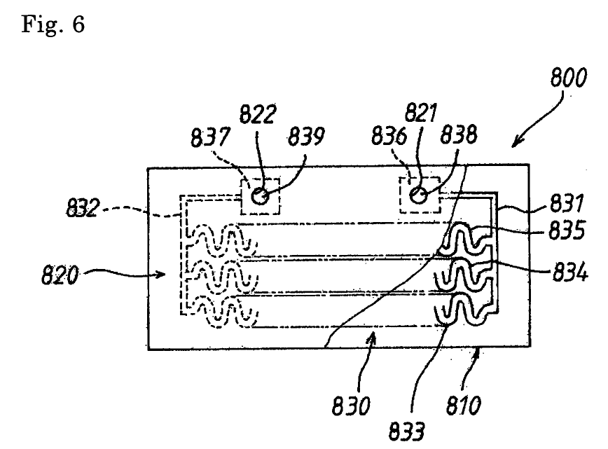

FIG. 6 shows the main part of a third embodiment of the invention. According to the third embodiment, a different heater section 800 is incorporated in the soot sensor according to the first embodiment, in the place of the heater section 400.

The heater section 800 performs a heat-cleaning function similarly to the heater section 400 of the first embodiment, and the heater section 800 is provided for the leading end side of the insulator 200 according to the first embodiment, through the entire circumference of the small diameter portion 232 of the leading end side part 230, similarly to the heater section 400.

The heater section 800 preferably includes two alumina sheets 810 and 820 and a heating member 830 as shown in FIG. 6. The heating member 830 has lead sections 831 and 832, three heat element sections 833, 834 and 835 and positive and negative side electrode pads 836 and 837. The lead sections 831 and 832 face toward each other in an L-shaped configuration along the inner surface of the alumina sheet 810, as shown in FIG. 6.

The three heat element sections 833, 834 and 835 are displayed in parallel with each other along the inner surface of the alumina sheet 810 between the lead sections 831 and 832. Both ends of the heat element sections 833, 834 and 835 are connected to the lead sections 831 and 832. According to the third embodiment, the heat element sections 833, 834 and 835 have a waveform pattern having alternate upper projections and lower projections, as shown in FIG. 6.

The positive and negative electrode pads 836 and 837 are provided on the inner surface of the alumina sheet 810 through the facing ends of the lead sections 831 and 832. The heating member 830 is preferably formed by firing a platinum paste, similarly to the heating member 430.

The inner surface of the alumina sheet 820 is press-fitted to the inner surface of the alumina sheet 810 through the heating member 830. The parts corresponding to the center parts of the electrode pads 836 and 837 of the alumina sheet 820 have through-holes 821 and 822.

The through-holes 821 and 822 have via-holes 838 and 839 preferably formed by firing a platinum paste.

### Fourth Embodiment

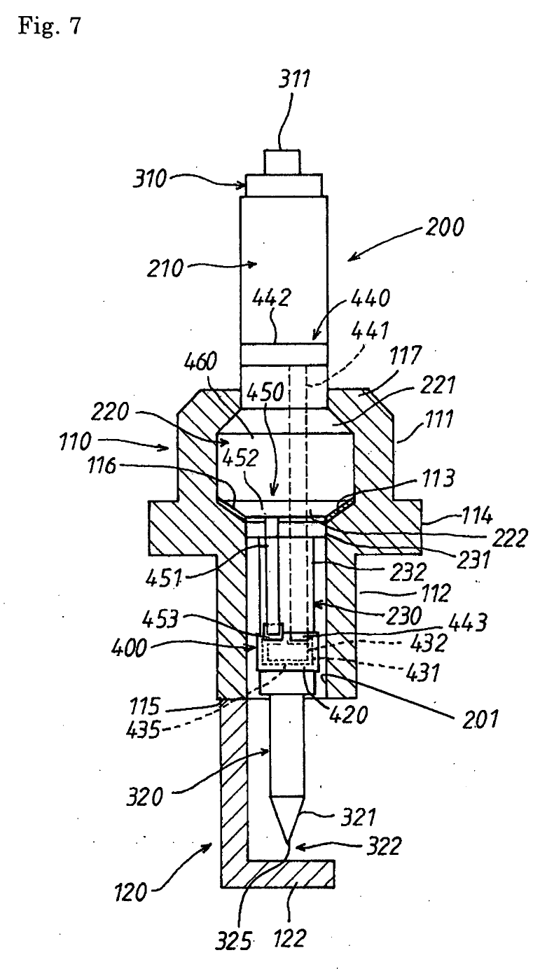

FIG. 7 shows a fourth embodiment of the invention. According to the fourth embodiment, the metal housing 110 according to the first embodiment is modified as follows.

First, the axial length of the metal housing 110 is long enough to surround the small diameter part or portion 232 of the leading end side part 230 of the insulator 200 including the leading end, as shown in FIG. 7. With this, construction the axial length of the outer electrode 120 is made shorter by the amount equal to the increase in axial length of the metal housing 110. The rest of the construction is the same as that of the first embodiment.

According to the fourth embodiment, with this construction, the metal housing 110 is long enough to surround the small diameter part 232 of the leading end side part 230 of the insulator 200 including the leading end, as described above. Thus, the leading end side part 230 of the insulator 200, including the leading end portion of the small diameter part 232, is positioned inside of the metal housing 110. Therefore, soot cannot easily spread within the metal housing 110, and the leading end side part 230 of the insulator 200 is not readily exposed to the soot. As a result, according to the fourth embodiment, the operational effects according to the first embodiment can be obtained, by effectively isolating the leading end side part 230 of the insulator 200 from soot.

It will be appreciated that the invention is not limited to the embodiments described above, and, in this regard, the present invention can be implemented with various modifications including the following:

(1) The form of each of the heat element sections of the heater is not limited to the patterns or forms of each of the heat element sections of the heater section 400 or 800, and may be changed as required.

(2) The part at which the insulator is positioned within the leading end side part of the center electrode is not required to face toward the heater.

(3) The heater may be provided as a part of the entire circumference of the leading end side part of the insulator, rather than the entire circumference.

(4) A discharge gap may be constructed between the internal wall of the pipe where the soot sensor is placed and the center electrode, and the outer electrode may be eliminated.

This application is based on Japanese Patent Application JP 2006-129771, filed May 9, 2006, the entire content of which is hereby incorporated by reference, the same as if fully set forth herein.

Although the invention has been described above in relation to preferred embodiments and modifications thereof, it will be understood by those skilled in the art that other variations and modifications can be effected in these preferred embodiments without departing from the scope and spirit of the invention.

What is claimed is:

1. A soot sensor comprising:
an insulator having a through-hole therein;
a center electrode provided in the through-hole of the insulator so that a leading end of the center electrode protrudes from a leading end of the insulator and faces a discharge gap; and
a heating member embedded in the insulator and having a leading end,
wherein the leading end of the heating member is spaced from the leading end of the center electrode by at least 10 mm.

2. The soot sensor according to claim 1, wherein:
a surface of the insulator extending between the leading end of the insulator and the leading end of the heating member has a length of about 3 mm to 12 mm.

3. The soot sensor according to claim 1, wherein the insulator has a thickness of from about 0.7 mm to 3 mm at a part thereof between the heating member and the center electrode.

4. The soot sensor according to claim 1, further comprising a metal housing surrounding a peripheral portion of the insulator, the leading end of the insulator being positioned within the metal housing.

5. The soot sensor according to claim 4, further comprising an outer electrode connected to the metal housing and having a tip section disposed opposite the leading end of the center electrode across a discharge gap.

6. The soot sensor according to claim 4, wherein the metal housing provides a common ground for the center electrode and the heating member.
